(19) United States
(12) Patent Application Publication
(10) Pub. No.: US 2005/0064489 A1
(43) Pub. Date: Mar. 24, 2005

(54) Engineered U6 and H1 promoters

(76) Inventors: Fang Liang Zhang, Fanwood, NJ (US); Luquan Wang, East Brunswick, NJ (US)

Correspondence Address: Albert Wai-Kit Chan;Law Offices of Albert Wai-Kit Chan LLC, World Plaza, Suite 604, 141-07 20th Avenue, Whitestone, NY, 11357, US

(21) Appl. No.: 10/947,035

(22) Filed: Sep. 22, 2004

Related U.S. Application Data
(60) Provisional application No. 60/505,677, filed on Sep. 24, 2003.

(51) Int. Cl. C12Q 1/68; A01K 67/00; C07H 21/02; C12N 15/85

(52) U.S. Cl. 435/6; 435/455; 435/320.1; 435/325; 800/8; 536/23.1

(57) ABSTRACT

Several engineered U6 and H1 promoters have been discovered. Using these engineered U6 and H1 promoters, anti-sense or siRNA can be expressed in a regulated way. In the absence of an inducer, the antisense or siRNA is not expressed by the promoters. In the presence of an inducer, the antisense or siRNA can be expressed by the promoters.

This application claims benefit of U.S. Provisional Application No. 60/505,677, Filed Sep. 24, 2003 the content of which is incorporated by reference here into this application.

The present application includes a Sequence Listing filed herewith on a floppy disk. The Sequence Listing is presented in a single file named sequence.txt, and having 4,947 bytes, the disclosure of which is incorporated herein by reference in its entirety.

Throughout this application, various references are referred to. Disclosures of these publications in their entireties are hereby incorporated by reference into this application in order to more fully describe the state of the art to which this invention pertains.

### BACKGROUND OF THE INVENTION

RNAi (RNA interference) is a phenomenon that small double-stranded RNA (Referred as small interference RNA or siRNA) can knock down the expression of its corresponding gene. RNAi has been observed in plant, Celegans and Drosophila long time ago. It was until recently that RNAi was discovered to work in mammalian system [1].

Small interference RNA (siRNA) is 19-29 nt double-stranded RNA. It works by cleaving and destroying its cognate RNA. siRNA first assembles into RNA-induced silencing complexes (RISCs), and it then activates the complex by unwinding its RNA strands. The unwound RNA strands subsequently guide the complex to the complementary RNA molecules, where the complex cleaves and destroys the cognate RNA, which results in RNAi phenomenon [2, 3, 4, 5].

siRNA can be obtained directly by chemical synthesis. Briefly, two single stranded and complimentary RNAs (19-29 nt) are chemically synthesized. The synthesized RNAs are then annealed together in vitro to form double stranded small interference RNA (siRNA). siRNA can be delivered into the cell using transfection and electroporation.

siRNA can also be synthesized by in vitro transcription. Briefly, two DNA fragments which each encode one promoter and a siRNA target sequence (19-29 nt) can be transcribed into RNAs by using in vitro transcription reaction. The RNAs can be purified and annealed together using standard methodology to form siRNA duplex. The siRNA can be transfected into cells to generate silencing effect. Alternatively, one DNA fragment which encodes a promoter and a short hairpin DNA (about 70 bp) can be transcribed into a small hairpin RNA. The RNA can be purified and transfected into the cell to generate siRNA effects.

One novel way of generating siRNA is to use DNA vector-based siRNA approach. Here a small DNA insert (about 70 bp) encoding a short hairpin RNA targeting the gene of interest is cloned into a plasmidic or viral vector containing a promoter, in particular U6 or H1 promoter. The insert-containing vector can be transfected into the cell, and it expresses the short hairpin RNA. The hairpin RNA is rapidly processed by the cellular machinery into 19-29 nt double stranded RNA (siRNA). This approach is also named vector-based siRNA approach [2, 3, 4, 5, 6].

Yet another novel way of generating siRNA is “siRNA cassette” based technology. “siRNA cassette” is a PCR product which consists of a promoter (e.g. U6 and H1 promoter) and terminator sequence flanking a DNA insert encoding a hairpin siRNA (about 70 bp). After transfected into cells, the DNA insert encoding a hairpin siRNA is expressed from the PCR product, and generating siRNA [7, 8]

Chemical synthetic siRNA and in vitro transcription siRNA approaches are RNA-based since they prepare siRNA in vitro and introduce prepared siRNA into cells. DNA Vector-based siRNA and siRNA cassette are DNA-based techniques since they introduce DNA into cells, and it relies on the cellular machinery to make siRNA.

In DNA-based siRNA technology and siRNA cassette technology, U6 and H1 promoters have been shown to be able to express siRNA in mammalian cells and generate siRNA effects. The U6 and H1 promoter utilizes the RNA polymerase III system. Since the expression of RNA polymerase III system is constitutive and ubiquitous, the expression of siRNA may bring un-wanted effects to the cells. Therefore, a regulated RNA polymerase III system for siRNA expression in mammalian system is not well established [9, 10, 11].

### SUMMARY OF THE INVENTION

This invention is based on the discovery of several engineered U6 and H1 promoters. These promoters can express genes in a regulated way. In the presence of inducers, the gene can be expressed. In the absence of inducers, the gene expression is turned off.

The term “engineered” as in an engineered promoter, indicates that the promoter is not found in nature, in that all or a portion of the nucleic acid sequence of the promoter is created or selected by man. The term “promoter” means a nucleic acid sequence that is sufficient to direct transcription.

In particular, the engineered U6 and H1 promoters are created by incorporating a tetracycline operator sequence (TetO) into the native U6 and H1 promoter sequence. The tetracycline operator sequence (TetO) is placed either upstream or downstream of the TATA box of U6 or H1 promoter. Different tetracycline operator sequences (TetO) can be used. The tetracycline operator sequences are also tailored to fit the promoter position. When tetracycline repressor protein (TetR) is present, it binds the tetO site and blocks the transcription. In the presence of tetracycline, tetracycline binds TetR and changes its conformation and releases the TetO site. In this way, the U6 and H1 promoters can be regulated.

Tetracycline can be substituted by its analogs. This includes but is not limited to doxycycline. The concentration of tetracycline can be varied among a wide range from 0 to 1 mg/ml.

The variants of engineered U6 and H1 promoters can be prepared by mutagenesis of deletion, insertion or substitution as long as the mutation does not affect the transcription activity of engineered U6 and H1 promoters.

Different variant of Tet operon can be used for the engineered U6 and H1 promoters. The Tet operon sequence can be further truncated as long as it binds the TetR suppressor protein. Tet operon sequence can be increased to increase the binding affinity between TetO and TetR.

Engineered U6 and H1 promoters from different mammalian species can be prepared in a similar way. The engineered U6 and H1 promoter can be prepared based on the wild type U6 and H1 promoters from human, rat, mouse, pig or other mammalian species.

Engineered U6 or H1 promoters can be used to express any genes in mammalian system. These genes include but not limited to bacterial, fungi, plant, and mammalian genes. These genes can be either natural or artificial. “Artificial genes” means that the genes do not exist in nature but are created or designed by human.

In particular, engineered U6 and H1 promoters can be used to express siRNA. A short hairpin DNA insert (about 70 bp) encoding a short hairpin RNA (“siRNA precursor”) targeting the gene of the interest is placed under the promoters, and a termination signal (four to six Ts) is placed after the short DNA insert. The fragment containing engineered U6 or H1 promoters, the short DNA insert and the termination signal is named as “siRNA cassette”. A siRNA cassette can be transfected directly into mammalian cells, where the hairpin RNA is expressed and processed by cellular machinery into 19-29 nt double stranded RNA (siRNA). Alternatively, a siRNA cassette can be cloned into a plasmid vector. The plasmid can then be transfected into cells, where the hairpin RNA is expressed and processed by cellular machinery into 19-29 nt double stranded RNA (siRNA). In another aspect, a siRNA cassette can be cloned into a viral vector, and a virus can be made from the viral vector. The virus can then be used to infect cells to express siRNA. The virus include but not limited to adenovirus, retrovirus, and lentivirus.

The term “siRNA precursor” indicates a nucleic acid sequence that encodes a ribonucleic acid (RNA) precursor, wherein the precursor comprises the following: (i) a first stem portion comprising sequence of at least 18 nucleotids that is complementary or identical to a sequence of a messenger RNA (mRNA) of a target genes; (ii) a second stem portion comprising a sequence of at least 18 nucleotides that is sufficiently complementary to the first stem portion to hybridize with the first stem portion to form a duplex stem; and (iii) a loop portion at least 4 nucleotide that connects the two stem portions.

The target for siRNA can be any gene. These genes can be either natural genes or artificial genes. The genes can be but not limited to be bacterial, fungal, viral, plant, and mammalian genes. For the siRNA constructs containing engineered U6 or H1 promoters, the form of delivery to mammalian cell or organisms can be in any suitable form, either as liposomal, viral, or other forms. siRNA containing engineered U6 or H1 promoter can be delivered into different mammalian hosts. This includes but not limited to rat, mouse, and human.

siRNA containing engineered U6 or H1 promoters can be used for gene therapy. For example, siRNA containing engineered U6 or H1 promoter can target oncogenes, and suppress the expression of the oncogenes, which may result in the inhibition of tumor growth.

Engineered U6 and H1 promoters have a lot advantages over the wild type U6 and H1 promoters. Engineered U6 and H1 promoters can express genes in a regulated way. In contrast, the native U6 and H1 promoters (the prior art) express genes in a constitutive way. A regulated promoter is particularly useful in siRNA expression. By using engineered U6 and H1 promoters, siRNA expression can be well controlled. If the siRNA expression is needed, the U6 and H1 promoters can be turned on by an inducer. If the siRNA expression is not needed, the U6 and H1 promoter can be turned off by removing the inducer.

When the target genes are essential for cell survival, stable cell line transfected with siRNA vector against the target genes cannot be established because constitutive expression of siRNAs may be lethal to the cells. However, by using engineered U6 and H1 promoters, stable cell line transfected with inducible siRNA vector against the target genes can be established because siRNA can be turned off. These stable cell lines can then be studied by inducing the siRNA expression via adding an inducer.

When the target genes are essential for cell survival, transgenic animals bearing siRNA expression vector against the target genes cannot be established because constitutive expression of those siRNAs may be lethal to the embryos. However, by using engineered U6 and H1 promoters, transgenic animal transfected with inducible siRNA vector against the target genes can be established because siRNA can be turned off. These transgenic animals can then be studied by inducing the siRNA expression via adding an inducer.

Engineered U6 and H1 promoters are also useful in therapeutic siRNA. Long term expression of siRNA may be toxic to animal or human. By using engineered U6 and H1 promoters, the expression of siRNA can be regulated according to the requirement. The siRNA expression can be minimized to maintain the therapeutic effect while reducing the toxicity.

### DETAILED DESCRIPTION OF THE FIGURES

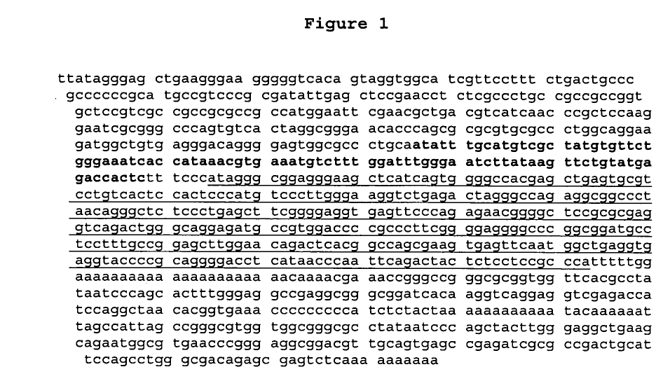

FIG. 1. Wild-type human H1 promoter and H1 RNA gene sequence (Genbank accession number X16612).

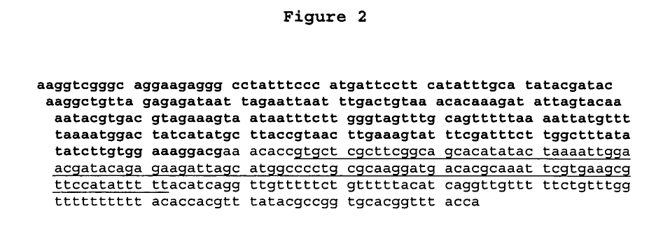

FIG. 2. Wild-type human U6 promoter and U6 RNA gene sequence (Genbank accession number X07425).

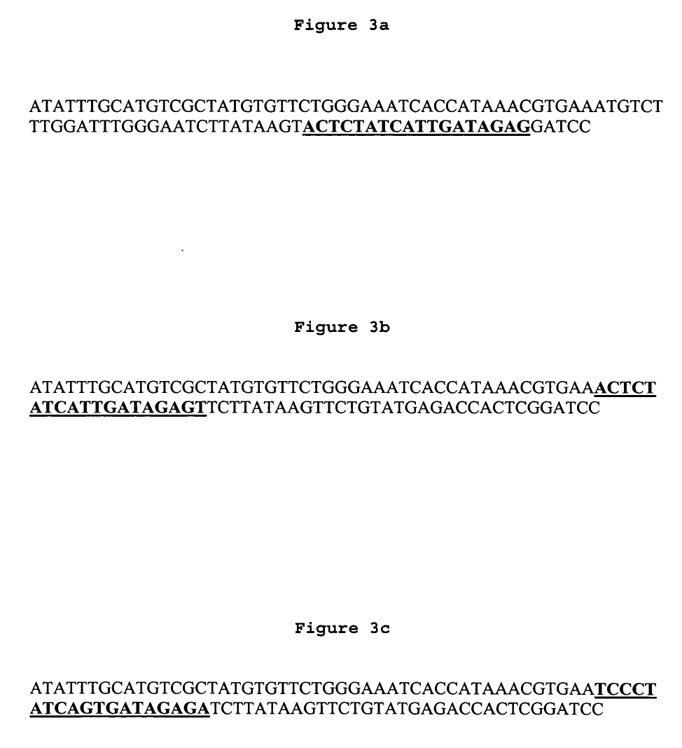

FIG. 3: a. Sequence of the engineered H1 promoter H1-IND-01. b. Sequence of the engineered H1 promoter H1-IND-02. c. Sequence of the engineered H1 promoter H1-IND-03.

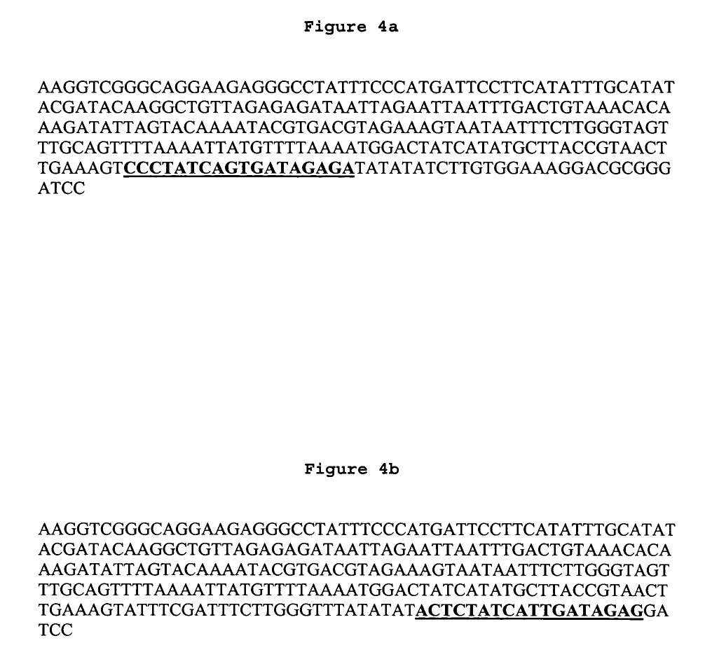

FIG. 4. a. Sequence of the engineered U6 promoter U6-IND-02. b. Sequence of the engineered U6 promoter U6-IND-03.

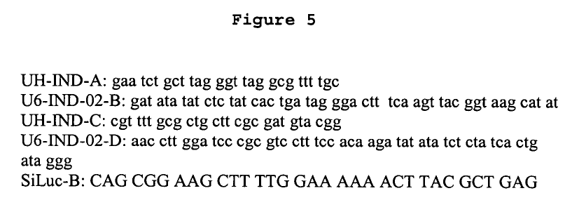

FIG. 5. PCR primers for construction of U6-IND-02 promoter and construct.

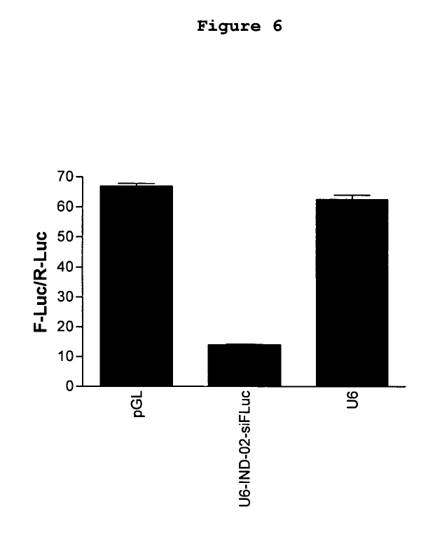

FIG. 6. Expression of siRNA by engineered U6 promoter. All the activities are normalized by Renilla Luciferase activity. The activities come from:
pGL: 293SFM cells transfected with pGL3-control (0.16 μg) and pRL-TK (0.16 μg).
U6-IND-02-siLuc: 293SFM cells transfected with pGL3-control (0.16 μg), pRL-TK (0.16 μg), and 1.6 μg of pRNA-U6-IND-02/Neo/siFLuc.
U6: 293SFM cells transfected with pGL3-control (0.16 μg), pRL-TK (0.16 μg), and 1.6 μg of pRNA-U6.1/Neo empty vector (Cat. SD1 201 from Genscript).

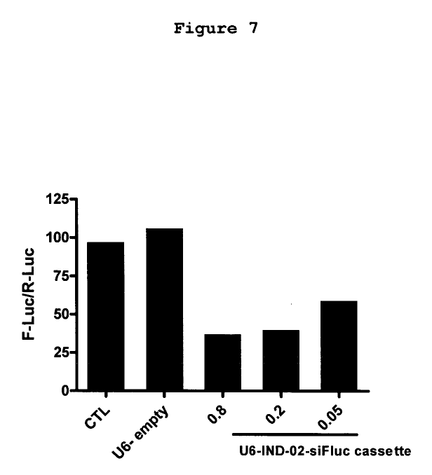

FIG. 7. Expression of siRNA cassette by engineered U6 promoter. All the activities are normalized by Renilla Luciferase activity. The activities come from:
CTL: 293-H cells transfected with 0.16 μg of pGL3-control and 0.16 μg of pRL-TK plasmid
U6-empty: 293-H cells transfected with pGL3-control (0.16 μg), pRL-TK (0.16 μg), and 1.6 μg of pRNA-U6.1/Neo empty vector (Cat. No. SD1201 from Genscript).
U6-IND-02-siFLuc cassette: 293-H cells transfected with pGL3-control (0.16 μg), pRL-TK (0.16 μg), plus 0.8 μg, 0.2 μg, or 0.05 μg of U6-IND-02 siFluc cassette.

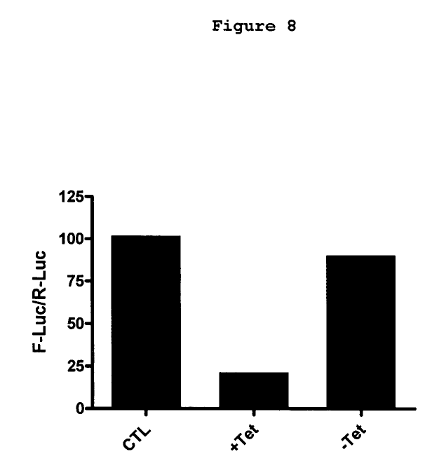

FIG. 8. Expression of siRNA by engineered U6-IND-02 in an inducible way. All the activities are normalized by Renilla Luciferase activity. The activities come from:
pGL: T-Rex 293 cells transfected with pGL3-control (0.16 μg) and pRL-TK (0.16 μg).
+Tet: T-Rex 293 cells transfected with p GL3-control (0.16 μg), pRL-TK (0.16 μg), and 1.6 μg of pRNA-U6-IND-02/Neo/siFLuc, in the presence of tetracycline of 5 μg/ml.
−Tet: T-Rex 293 cells transfected with pGL3-control (0.16 μg), pRL-TK (0.16 μg), and 1.6 μg of pRNA-U6-IND-02/Neo/siFLuc, in the absence of tetracycline.

### DETAILED DESCRIPTION OF THE INVENTION

U6 and H1 are small nuclear RNA genes. U6 and H1 RNA are transcribed by RNA pol III. The promoters for U6 and H1 have been defined (see FIGS. 1 and 2). H1 RNA is initiated at position 375, terminated at 715 (FIG. 1). The underlined in FIG. 1 is wild-type H1 promoter. U6 RNA are initiated at position 266, and terminated at 372 (FIG. 2). The underline in FIG. 2 is wild-type U6 promoter. Their termination sites contain multiple T. U6 and H1 promoters are constitutive RNA polymerase III promoters. It is the goal of this invention to establish regulated U6 and H1 promoters for mammalian systems.

Tetracycline-inducible system has been established for regulated expression of RNA pol II system, such as CMV promoter. In the CMV promoter, two tetracycline operator sequences (TetO2) have been inserted between the TATA box of the CMV promoter and the transcriptional start site. The TetO2 sequence itself has no effect on expression. When the tetracycline repressor protein (TetR) is present, it effectively binds the TetO2 sites and blocks transcription initiation. Tetracycline added to the culture medium binds to, and changes the conformation of, the TetR protein. This causes the TetR protein to release the TetO2 sites, de-repressing transcription from the CMV promoter. The result is high-level expression of the genes of interest. Expression levels can be modulated based on the tetracycline concentration, and the expression level can be reduced to background by removing tetracycline or increased to levels that are achieved with constitutive CMV expression vectors.

In contrast to RNA pol II system, regulated pol III mammalian system is not well established. Here we have developed a series of regulated U6 and H1 promoters (FIGS. 3 and 4). As shown in FIG. 3a, H1-IND-01 promoter is an engineered H1 promoter where a truncated TetO1 operon is placed downstream of the TATA box of H1 promoter. In FIG. 3b, H1-IND-02 promoter is another engineered H1 promoter where a truncated TetO1 operon is placed upstream of the TATA box of the H1 promoter. In FIG. 3c, H1-IND-03 promoter is an engineered H1 promoter where a truncated TetO2 operon is placed upstream of the TATA box of H1 promoter.

As shown in FIG. 4a, U6-IND-02 is an engineered U6 promoter where a truncated of TetO2 operon is placed upstream of TATA box of U6 promoter. In FIG. 4b, U6-IND-03 is another engineered U6 promoter where a truncated TetO1 operon is placed downstream of TATA box of U6 promoter.

The variants of engineered U6 and H1 promoters can be prepared by mutagenesis of deletion, insertion, or substitution as long as the mutation does not affect the transcription activity of engineered U6 and H1 promoters.

Different variant of Tet operon can be used for the engineered U6 and H1 promoters. The Tet operon sequence can be further truncated as long as it binds the TetR suppressor protein. Tet operon sequence can also be increased to increase the binding affinity between TetO and TetR.

Engineered U6 and H1 promoters from different mammalian species can be prepared in a similar way. The engineered U6 and H1 promoter can be prepared based on the wild type U6 and H1 promoters from human, rat, mouse, pig or other mammalian species.

Engineered U6 and H1 promoter will direct RNA expression in a regulated way. The TetO1 or TetO2 itself has minimal effect on expression. When the tetracycline repressor protein (TetR) is present, it effectively binds to TetO2 or TetO1 sites and blocks transcription initiation. When tetracycline is added, tetracycline binds to, and changes the conformation of the TetR protein. This change causes the TetR protein to release the TetO1 or TetO2 sites, de-pressing transcription from U6 and H1 promoter. The result is high-level expression of the genes of interest. Expression levels can be modulated based on the tetracycline concentration, and the expression level can be reduced to background by removing tetracycline or increased to levels that are achieved with constitutive U6 or H1 expression vectors.

Engineered U6 and H1 promoters can be used to express different genes in a regulated way. These genes could be artificial genes, or natural genes. The genes are placed downstream of engineered U6 or H1 promoters, together with a termination signal. The termination signal may be poly T or other appropriate signal.

One class of genes may be anti-sense genes or RNA. The anti-sense genes are artificial genes which complement with known genes. The anti-sense RNA can bind to its corresponding mRNA, which can result in the damage of mRNA and suppress the expression of its corresponding genes. Anti-sense RNA can be used for gene therapy for cancer or other related genes.

Another class of genes that engineered U6 or H1 promoter can express are small interference RNA (siRNA). A DNA insert about 70 bp which codes a small hairpin RNA (“siRNA precursor”) can be placed downstream of an engineered U6 and H1 promoter, together with a RNA pol III termination signal (four to six T).

The term “siRNA precursor” indicates a nucleic acid sequence that encodes a ribonucleic acid (RNA) precursor, wherein the precursor comprises the following: (i) a first stem portion comprising sequence of at least 18 nucleotids that is complementary or identical to a sequence of a messenger RNA (mRNA) of a target genes; (ii) a second stem portion comprising a sequence of at least 18 nucleotides that is sufficiently complementary to the first stem portion to hybridize with the first stem portion to for a duplex stem; and (iii) a loop portion at least 4 nucleotide that connects the two stem portions.

A vector which comprises an engineered U6 or H1 promoter and a RNA termination signal can be constructed according to standard procedures. The vector can be used to clone a small DNA insert encoding a small hairpin RNA to downstream of U6 and H1 promoter. The vector can be introduced into mammalian cells to express small hairpin RNA, which can be processed into small RNA duplex (siRNA). The siRNA can silence the expression of the target gene.

The vectors can be constructed by recombinant DNA techniques known in the art. Vectors can be plasmid, viral, retroviral, or other vectors known in the art such as those described herein, used for replication and expression in mammalian cells or other targeted cell types. The nucleic acid sequence encoding the siRNA can be prepared using known techniques in the art.

Alternatively, a DNA fragment consists of an engineered U6 and H1 promoter, siRNA insert and termination signal can be prepared using PCR or enzymatic method or other known techniques in the art (i.e. siRNA cassette). The DNA fragment can be delivered into mammalian cells using in vitro or in vivo methods. The DNA fragment can express the small hairpin RNA, which can be processed into small RNA duplex (siRNA). The siRNA can suppress the expression of the target gene.

As indicated above, engineered U6 and H1 promoters can direct synthesis of small hairpin RNA and generate siRNA effect. The stem sequence is complementary or identical to a target sequence. The target sequence can be either artificial sequence or natural sequence. The target sequence can also be either bacterial, viral, retroviral, plant, animal, or human sequences.

siRNA can be delivered to different hosts. These hosts include, but are not limited to, mammalian cells, animal or human. The delivery form can be chosen whichever is appropriate (e.g. plasmid or viral fragment). They can be delivered using different methods such as lipofectamine, or electroporation or injection.

siRNA transgenic animal and plant can be produced by known arts using engineered U6 and H1 promoters.

siRNA can suppress the expression of its corresponding gene. siRNA can have different applications. A siRNA against a virus may be used to eliminate a virus infection. A siRNA against a tumor gene may be used to suppress tumor growth. A siRNA against a disease can be used for the therapy of a disease.

Assay for testing engineered U6 and H1 promoters:

Luciferase assay can be used to test whether engineered U6 and H1 promoters can express siRNA. A know siRNA targeting firefly luciferase has been described in literature [6]. The target sequence is ctt acg ctg agt act tcg a, which corresponds to 434-452 in p GL-3 control vector. p GL-3 control vector contains a firefly luciferase gene. An insert which includes both the sense strand and anti-sense strand of the target sequence, a loop and poly(T) termination signal can be prepared. The insert sequence is the following:

BamH I
HindiII
GGATCCCGCTTACGCTGAGTACTTCGATTCAAGAGATCGAAGTACTCAGCGTAAGTTTTTTCCAAAAGCTT
| Sense | Loop | antisense | Termination

This insert sequence can then placed under the control of an engineered U6 or H1 promoter. The resulting vector or fragment can be transfected into a mammalian cell line such as HEK293 cells together with pGL-3 control vector, and pRL-TK vector. pGL-3 control vector encodes firefly luciferase, while pRL-TK vector encodes Renilla Luciferase. If siRNA expresses, the activity of firefly luciferase will be inhibited while Renilla Luciferase activity is unchanged. The activity of firefly luciferase activity can then be normalized for transfection efficiency using Renilla Luciferase. The activity of firefly and Renilla luciferase activity can be easily detected using Dual Luciferase Assay kit from Promega (Cat # E1910) in Luminescence detection instrument.

This invention will be better understood from the examples which follow. However, one skilled in the art will readily appreciate that the specific methods and results discussed are merely illustrative of the invention as described more fully in the claims which follow thereafter.

### EXAMPLES 1

### Construction of U6-IND-O2 Promoter and U6-IND-02 Containing Vector

The primers for constructing U6-IND-02 promoter and U6-IND-02 containing vectors are listed in FIG. 5.
a. Using UH-IND-A and U6-IND-02-B primer to perform a PCR reaction on pRNA-U6.1/Neo (Genscript, Cat# SD1201) using pfu enzyme. Purify the PCR product.
b. Take 0.5 μl of PCR product from first step, using UH-IND-C and U6-IND-02-D primers, perform a PCR reaction using pfu enzyme. Purify the PCR product.
c. Cut the PCR product from the second step with MluI and BamH I, gel purify to get the insert fragment.
d. Cut the pRNA-U6.1/Neo/siFLuc (Genscript, Cat #SD1501) with MluI and BamH I to get the vector fragments.
e. Ligate the insert fragment into the vector fragment, sequencing to verify the construct using T7 and BGH reverse primer.
f. The engineered promoter is U6-ND-02 promoter as shown in FIG. 4a. The construct is named pRNA-U6-IND-02/neo/siFluc. g. The engineered promoters U6-IND-03, H-IND-01, H1-IND-02, and H1-IND-03 can be constructed using similar methods.

### EXAMPLE 2

### Expression of siRNA by Engineered U6-Vector

In pRNA-U6-IND-02/neo/siFluc construct, an insert encoding the siRNA against Firefly luciferase is placed under the control of U6-IND-02 promoter.
a. To observe the effect of siRNA effect by engineered U6-promoter U6-IND-02, three sets of transfections are needed: a. pGL-control and pRL-TK (Promega, Cat. #E2241) vector alone; b. pGL-3 control (Promega, Cat. #E1741), pRL-TK, and pRNA-U6-IND-02/neo/siFluc; c. pGL-3 control, pRL-TK, and an empty pRNA vector pRNA-U6.1/Neo (SD1201 from Genscript).
b. For cell transfection, 12-well plates can be used. For 293SFM cell (Cat.# 11625-019) from Invitrogen, 20,000 cells can be seeded the day before transfection.
c. The amount pRNA-U6-IND-02/neo/siFluc used for transfection should be 10-30 folds higher than that of pGL-3 control plasmid. For 293SFM, 0.16 μg of pGL-3 control and 0.16 μg pRL-TK vector were used, 1.6 μg of pRNA-U6-IND-02/neo/siFluc construct or empty vector are used for each well.
d. The plasmid can be transfected into mammalian cells using Lipofectamine™-2000 following the protocol.
e. The Firefly and Renilla luciferase activities can be measured using Dual Luciferase assay kit from Promega (Cat. #E1910) after 24 hrs of transfection.
f. The activities of Firefly luciferase are normalized using Renilla luciferase activity.
g. As shown in FIG. 6, U6-IND-02 can express the siRNA for Firefly luciferase. The siRNA can inhibit the Firefly luciferase activity by about 80%.

### EXAMPLE 3

### Expression of siRNA Cassette by Engineered U6 Promoter

a. siRNA cassette can be prepared either by enzyme digestion or PCR reactions. For enzyme digestion method, 100 μg of pRNA-U6-IND-02/neo/siFluc plasmid can be digested using Mlu I and Hind III. The digested products can then be run on a gel, and the smaller fragments can be purified. The smaller fragment contains U6-IND-02 promoter, an insert coding siRNA against firefly luciferase, and a poly(T) termination signal. This fragment is a siRNA cassette. For PCR method, two oligos (UH-IND-A and siLuc-B in FIG. 5) can be used to perform a PCR reaction using pRNA-U6-IND-02/neo/siFluc. The PCR product can be then purified by Qiagen PCR purification kit. The purified PCR product is a siRNA cassette.
b. To observe the silencing effect of siRNA cassette using U6-IND-02 promoter, five sets of transfection are needed: a). pGL-3 control (Promega, Cat. #E1741) and pRL-TK (Promega, Cat. #E2241) vector alone. b). pGL-3 control, pRL-TK, and an empty pRNA vector pRNA-U6.1/Neo (Genscript, Cat. No. SD1201); c). pGL-3 control, pRL-TK, and 0.8 μg of siFLuc/U6-IND-02 cassettes. d). pGL-3 control, pRL-TK, and 0.2 μg of siFLuc/U6-IND-02 cassettes. e). pGL-3 control, pRL-TK, and 0.05 μg of siFLuc/U6-IND-02 cassettes.
c. For cell transfection, 12-well plates can be used. For 293-H cell from Invitrogen, 30,000 cells can be seeded the day before transfection.
d. Different amount of siLuc cassette can be tested. For 293-H, 0.16 μg of pGL-3 control and 0.16 μg pRL-TK vector are used, 0.05 μg to 0.8 μg of siFluc/U6-IND-02 cassette are used for each well. 1.6 μg of pRNA-U6.1/Neo is used.
e. The plasmid can be transfected into mammalian cells using Lipofectamine Plus™ following the protocol.
f. The Firefly and Renilla luciferase activities can be measured using Dual Luciferase assay kit from Promega (Cat. #E1910) after 36 hrs of transfection.
g. The activities of Firefly luciferase need to be normalized using Renilla luciferase activity.
h. As shown in FIG. 7, siRNA cassette using engineered U6-IND-02 can inhibit firefly luciferase activity by about 70%.

### EXAMPLE 4

### Expression of siRNA in a Regulated Way

In pRNA-U6-IND-02/neo/siFluc construct, an insert encoding the siRNA against Firefly luciferase is placed under the control of U6-IND-02 promoter. U6-IND-02 can express siRNA in a regulated way.
a. To observe the effect of siRNA in a regulated way, T-Rex 293 cell (Invitrogen Cat.# 11625-019) can be used. T-Rex 293 cell is a stable cell line with TetR repressor protein expression. Three sets of transfections are needed in T-Rex 293: a). pGL-control and pRL-TK (Promega, Cat. #E2241) vector alone; b). pGL-3 control (Promega, Cat. #E1741), pRL-TK, and pRNA-U6-IND-02/neo/siFluc, in the absence of tetracycline; c). pGL-3 control (Promega, Cat. #E1741), pRL-TK, and pRNA-U6-IND-02/neo/siFluc, in the presence of tetracycline (5 μg/ml).
b. For cell transfection, 12-well plates can be used. 20,000 T-Rex 293 cell can be seeded the day before transfection.
c. The amount pRNA-U6-IND-02/neo/siFluc used for transfection should be 10-30 folds higher than that of pGL-3 control plasmid. 0.16 μg of pGL-3 control and 0.16 μg pRL-TK vector were used, 1.6 μg of pRNA-U6-IND-02/neo/siFluc construct are used for each well.
d. The plasmid can be transfected into mammalian cells using Lipofectamine™-2000 following the protocol.
e. The Firefly and Renilla luciferase activities can be measured using Dual Luciferase assay kit from Promega (Cat. #E1910) after 24 hrs of transfection.
f. As shown in FIG. 8, in the absence of tetracycline, the siRNA is not expressed, and the activity of Firefly luciferase is similar to the control. In the presence of tetracycline (5 μg/ml), the siRNA is expressed. The siRNA can inhibit the Firefly luciferase activity by about 80%.

### References

1. Elbashir S M, Harborth J, Lendeckel W, Yalcin A, Weber K, Tuschl T. (2001) Duplexes of 21-nucleotide RNAs mediate RNA interference in cultured mammalian cells. Nature 411: 494-498.
2. Yu J Y, DeRuiter S L, Turner D L. (2002) RNA interference by expression of short-interfering RNAs and hairpin RNAs in mammalian cells. Proc Natl Acad Sci USA. 99(9):6047-6052.
3. Brummelkamp, T. R., Bernards, R., and Agami, R. (2002) A system for stable expression of short interfering RNAs in mammalian cells. Science 296: 550-553.
4. Jacque, J.-M., Triques, K., and Stevenson, M. (2002) Modulation of H1V-1 replication by RNA interference. Nature 418: 435-438.
5. Sui, G., Soohoo, C., Affar, E. B., Gay, F., Shi, Y., Forrester, W. C., and Shi, Y. (2002) A DNA vector-based RNAi technology to suppress gene expression in mammalian cells. Proc. Natl. Acad. Sci. USA 99(8): 5515-5520.
6. Lewis, D. L., Hagstrom, J. E., Loomis, A. G., Wolff, J. A., Herweijer, H. (2002) Efficient delivery of siRNA for inhibition of gene expression in postnatal mice. Nat Genet. 2002 September;32(1):107-8.
7. Castanotto D, Li H, Rossi J J (2002) Functional siRNA expression from transfected PCR products. RNA. November;8(11): 1454-60.
8. Gou D, Jin N, Liu L. Gene silencing in mammalian cells by PCR-based short hairpin RNA. FEBS Lett. 2003 Jul. 31;548(1-3):113-8.
9. van de Wetering M, Oving I, Muncan V, Pon Fong M T, Brantjes H, van Leenen D, Holstege F C, Brummelkamp T R, Agami R, Clevers H. Specific inhibition of gene expression using a stably integrated, inducible small-interfering-RNA vector. EMBO Rep. 2003 June;4(6):609-15.
10. Matsukura S, Jones P A, Takai D. Establishment of conditional vectors for hairpin siRNA knockdowns. Nucleic Acids Res. 2003 Aug. 1;31(15):e77.
11. Ohkawa J, Taira K. Control of the functional activity of an antisense RNA by a tetracycline-responsive derivative of the human U6 snRNA promoter. Hum Gene Ther. 2000 Mar. 1;11(4):577-85.
12. Czaudema F, Santel A, Hinz M, Fechtner M, Durieux B, Fisch G, Leenders F, Arnold W, Giese K, Klippel A, Kaufmann J. Inducible shRNA expression for application in a prostate cancer mouse model. Nucleic Acids Res. 2003 Nov. 1;31(21):e127.

1-18. (Cancel)

19. An engineered H1 promoter selected from a group consisting of H1-IND-01 as shown in FIG. 3a and its variants, H1-IND-02 as shown in FIG. 3b and its variants, H1-IND-03 as shown in FIG. 3c and its variants, U6-IND-02 as shown in FIG. 4a and its variants, and U6-IND-03 as shown in FIG. 4b and its variants.

20. A vector comprising the promoter of claim 19.

21. A DNA fragment comprising the promoter of claim 19.

22. The DNA fragment of claim 21, wherein it is linear or circular.

23. A host cell containing the DNA of claim 21.

24. A transgenic organism comprising the promoter of claim 19, the DNA comprising an engineered H1 promoter selected from a group consisting of H1-IND-01 as shown in FIG. 3a and its variants, H1-IND-02 as shown in FIG. 3b and its variants, H1-IND-03 as shown in FIG. 3c and its variants, U6-IND-02 as shown in FIG. 4a and its variants, and U6-IND-03 as shown in FIG. 4b and its variants, or the cell comprising an engineered H1 promoter selected from a group consisting of H1-IND-01 as shown in FIG. 3a and its variants, H1-IND-02 as shown in FIG. 3b and its variants, H1-IND-03 as shown in FIG. 3c and its variants, U6-IND-02 as shown in FIG. 4a and its variants, and U6-IND-03 as shown in FIG. 4b and its variants.

25. A method of expressing siRNA by placing antisense gene under the control of the promoter of claim 19.

26. A method of expressing siRNA by placing small hairpin loop under the control of the promoter of claim 19.

27. A method of expressing siRNA by placing an siRNA precursor under the control of the promoter of claim 19.

28. A method of expressing a RNA fragment under the control of the promoter of claim 19.

29. The method of claim 28, wherein the RNA fragment is an antisense RNA, hairpin-loop RNA, or siRNA.

30. The target sequence of claim 19 is an animal gene, a viral gene, a bacterial gene, a plant gene, a yeast gene, a human gene, or an artificial gene.

31. The use of the siRNA generated by the promoter of claim 19.

32. A matrix containing the nucleic acid molecules of claim 19.


SEQUENCE LISTING (12 sequences)

SEQ ID NO: 1 - DNA, 1057 bp, Human
ttatagggagctgaagggaagggggtcacagtaggtggcatcgttcctttctgactgcccgccccccgcatgccgtcccgcgatattgagctccgaacctctcgccctgccgccgccggtgctccgtcgccgccgcgccgccatggaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactctttcccatagggcggagggaagctcatcagtggggccacgagctgagtgcgtcctgtcactccactcccatgtcccttgggaaggtctgagactagggccagaggcggccctaacagggctctccctgagcttcggggaggtgagttcccagagaacggggctccgcgcgaggtcagactgggcaggagatgccgtggaccccgcccttcggggaggggcccggcggatgcctcctttgccggagcttggaacagactcacggccagcgaagtgagttcaatggctgaggtgaggtaccccgcaggggacctcataacccaattcagactactctcctccgcccatttttggaaaaaaaaaaaaaaaaaaaaaacaaaacgaaaccgggccgggcgcggtggttcacgcctataatcccagcactttgggaggccgaggcgggcggatcacaaggtcaggaggtcgagaccatccaggctaacacggtgaaacccccccccatctctactaaaaaaaaaaaatacaaaaaattagccattagccgggcgtggtggcgggcgcctataatcccagctacttgggaggctgaagcagaatggcgtgaacccgggaggcggacgttgcagtgagccgagatcgcgccgactgcattccagcctgggcgacagagcgagtctcaaaaaaaaaa

SEQ ID NO: 2 - DNA, 464 bp, Homo sapiens
aaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagtttttaaaattatgttttaaaatggactatcatatgcttaccgtaacttgaaagtatttcgatttcttggctttatatatcttgtggaaaggacgaaacaccgtgctcgcttcggcagcacatatactaaaattggaacgatacagagaagattagcatggcccctgcgcaaggatgacacgcaaattcgtgaagcgttccatatttttacatcaggttgtttttctgtttttacatcaggttgtttttctgtttggttttttttttacaccacgtttatacgccggtgcacggtttacca

SEQ ID NO: 3 - DNA, 99 bp, Artificial
  Engineered H1 promoter
atatttgcatgtcgctatgtgttctgggaaatcaccataaacgtgaaatgtctttggatttgggaatcttataagtactctatcattgatagaggatcc

SEQ ID NO: 4 - DNA, 99 bp, Artificial
  Engineered H1 promoter
atatttgcatgtcgctatgtgttctgggaaatcaccataaacgtgaaactctatcattgatagagttcttataagttctgtatgagaccactcggatcc

SEQ ID NO: 5 - DNA, 99 bp, Artificial
  Engineered H1 promoter
atatttgcatgtcgctatgtgttctgggaaatcaccataaacgtgaatccctatcagtgatagagatcttataagttctgtatgagaccactcggatcc

SEQ ID NO: 6 - DNA, 265 bp, Artificial
  Engineered U6 promoter
aaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtccctatcagtgatagagatatatatcttgtggaaaggacgcgggatcc

SEQ ID NO: 7 - DNA, 265 bp, Artificial
  Engineered U6 promoter
aaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatactctatcattgatagaggatcc

SEQ ID NO: 8 - DNA, 27 bp, Artificial
  PCR primer
gaatctgcttagggttaggcgttttgc

SEQ ID NO: 9 - DNA, 50 bp, Artificial
  PCR Primer
gatatatatctctatcactgatagggactttcaagttacggtaagcatat

SEQ ID NO: 10 - DNA, 27 bp, Artificial
  PCR Primer
cgttttgcgctgcttcgcgatgtacgg

SEQ ID NO: 11 - DNA, 54 bp, Artificial
  PCR Primer
aaccttggatcccgcgtcctttccacaagatatatatctctatcactgataggg

SEQ ID NO: 12 - DNA, 33 bp, Artificial
  PCR Primer
cagcggaagcttttggaaaaaacttacgctgag